(19) United States
(12) Patent Application Publication
(10) Pub. No.: US 2024/0026591 A1
(43) Pub. Date: Jan. 25, 2024

(54) Washing Machine or Dishwasher for Cleaning Laundry or Dishes

(71) Applicant: Marco Begerok, Fulda (DE)

(72) Inventor: Marco Begerok, Fulda (DE)

(21) Appl. No.: 18/004,427

(22) PCT Filed: Jul. 5, 2021
(86) PCT No.: PCT/EP2021/068518

§ 371 Date: Jan. 5, 2023

(30) Foreign Application Priority Data
Jul. 9, 2020 (DE) 10 2020 004 138.8
Jul. 9, 2020 (DE) 20 2020 002 928.9

(51) Int. Cl. D06F 35/00; A47L 15/42; A61L 2/10

(52) U.S. Cl. CPC D06F 35/008; A47L 15/4242; A61L 2/10; A61L 2202/26; A61L 2202/122; A61L 2202/17

(57) ABSTRACT

A washing machine or dishwasher is disclosed. In an embodiment a washing machine or dishwasher includes a washing compartment configured to clean laundry or dishes and an ultraviolet radiation source configured to irradiate the laundry or the dishes, wherein the ultraviolet radiation source is arranged within the washing compartment.

This patent application is a national phase filing under section 371 of PCT/EP2021/068518, filed Jul. 5, 2021, which claims the priority of German patent application 10 2020 004 138.8, filed Jul. 9, 2020, and German patent application 20 2020 002 928.9 filed Jul. 9, 2020, each of which is incorporated herein by reference in its entirety.

### TECHNICAL FIELD

The present invention relates to a washing machine or dishwasher for cleaning laundry or dishes.

### BACKGROUND

It is known in the prior art, that viruses and bacteria can be killed with UV light.

UV-C light in particular is suitable for carrying out appropriate disinfection.

### SUMMARY

A washing machine or a dishwasher fulfill two tasks, on the one hand the laundry or the dishes should be optically cleaned. In addition, pathogenic germs, bacteria and viruses should be removed and/or killed. In particular, boiling water, i.e. water with a temperature of 100° Celsius, can be used for this purpose. However, operating a washing machine or dishwasher at 100° Celsius would damage the items to be washed, i.e. the laundry or dishes or plastic containers.

Embodiments provide a washing machine and a dishwasher which ensure complete and reliable killing of pathogenic germs, viruses and bacteria.

As a first embodiment of the invention a washing machine or a dishwasher for cleaning laundry or dishes is provided, wherein the washing machine or the dishwasher has a washing compartment for the laundry or the dishes, wherein the laundry or the dishes are cleaned within the washing compartment, wherein the washing machine or the dishwasher has an ultraviolet radiation source, in particular a UV-C radiation source, for irradiating the laundry or the dishes, and wherein the ultraviolet radiation source is arranged within the washing compartment.

Embodiments of the invention serves in particular to kill or combat pathogenic germs, bacteria and viruses, in particular corona viruses.

According to an exemplary embodiment of the invention, a washing machine or dishwasher is provided, wherein the ultraviolet radiation source is separated from the washing compartment by a first material, which is transparent to ultraviolet radiation, in particular UV-C radiation, and/or wherein the material is glass or plastic.

According to an exemplary embodiment of the invention, a washing machine or dishwasher is provided, wherein the first material is made of synthetic quartz glass.

According to a further exemplary embodiment of the present invention, a washing machine or dishwasher is provided, wherein the ultraviolet radiation source is formed to be movable, in particular turnable or rotatable, in order to scatter the ultraviolet radiation of the ultraviolet radiation source arbitrarily within the washing compartment during the washing process.

In a dishwasher in particular, in which the items to be washed are not moved during the washing process, it is useful for the ultraviolet radiation source to be moved in order to be able to irradiate uniformly the items to be washed.

According to a further exemplary embodiment of the present invention, a washing machine or dishwasher is provided, the washing machine or the dishwasher comprising:
a first ultraviolet radiation source illuminating the washing compartment from above, and/or
a second ultraviolet radiation source illuminating the washing compartment from below, and/or
a third ultraviolet radiation source illuminating the washing compartment from the left, and/or
a fourth ultraviolet radiation source illuminating the washing compartment from the right, and/or
a fifth ultraviolet radiation source, illuminating the washing compartment from the front, and/or
a sixth ultraviolet radiation source, illuminating the washing compartment from behind.

In a further embodiment according to the invention, a washing machine or dishwasher is provided, wherein a reflector is arranged in the washing compartment, so that ultraviolet radiation from the ultraviolet radiation source can be reflected back into the washing compartment.

With a reflector inside the washing compartment, the washing compartment can be illuminated with ultraviolet radiation more evenly.

In a further embodiment according to the invention, a washing machine or dishwasher is provided, wherein said reflector is formed to be movable, in particular rotating, in order to scatter the ultraviolet radiation arbitrarily during the washing process.

According to an exemplary embodiment of the invention, a washing machine or dishwasher is provided, wherein the washing compartment is lined with a second material reflecting ultraviolet radiation.

By lining the inner walls of the washing compartment with a material reflecting ultraviolet radiation, ultraviolet radiation can be reflected back into the washing compartment and thus uniform illumination of the items to be washed can be ensured.

In a further embodiment according the invention, a washing machine or dishwasher is provided, wherein the washing machine or dishwasher has a device for determining whether the washing compartment is locked, and wherein the ultraviolet radiation source can only be switched on, if the device signals a closed washing compartment.

In a further embodiment according to the invention, a washing machine or dishwasher is provided, wherein the ultraviolet radiation source is circular, semicircular or meander shaped and/or wherein between the outer area and the ultraviolet radiation source a third material absorbing ultraviolet radiation is arranged, and/or wherein the third material is glass, perforated sheet metal or plastic.

It can be considered an idea of the invention to provide a washing compartment for items to be washed, that is completely illuminated with an ultraviolet radiation, in particular with an UV-C radiation. In a particular embodiment, the ultraviolet radiation source interacts with a reflector, so that an ultraviolet radiation can be directed from anywhere within the washing compartment onto the items to be washed. Additionally, or alternatively, the ultraviolet radiation source can be formed to be movable, in particular rotatable.

The individual features may, of course, also be combined with one another, whereby in some cases beneficial effects surpassing the sum of the individual effects may result.

### BRIEF DESCRIPTION OF THE DRAWINGS

Further details and advantages of the invention become apparent from the exemplary embodiments illustrated in the drawings.

FIG. 1 shows a washing machine with a viewing window and an interior washing compartment, in which the laundry is cleaned;

FIG. 2 shows a washing machine with a circular ultraviolet radiation source within the washing compartment;

FIG. 3 shows an alternative washing machine with a meander shaped ultraviolet radiation source;

FIG. 4 shows a further alternative washing machine with a flat, meander shaped ultraviolet radiation source;

FIG. 5 shows a further washing machine with a circular ultraviolet radiation source;

FIG. 6 shows a cross section from above of a washing machine with an interstitial space between a material absorbing ultraviolet radiation, and a further material separating the ultraviolet radiation source from the items to be washed. The interstitial space is within the washing compartment;

FIG. 7 shows a cross section from above of an alternative washing machine, wherein the ultraviolet radiation source is arranged directly behind the viewing window of the washing machine;

FIG. 8 shows a dishwasher, within the washing compartment of which a meander shaped ultraviolet radiation source is arranged; and

FIG. 9 shows an alternative dishwasher with a rotatable ultraviolet radiation source.

### DETAILED DESCRIPTION OF ILLUSTRATIVE EMBODIMENTS

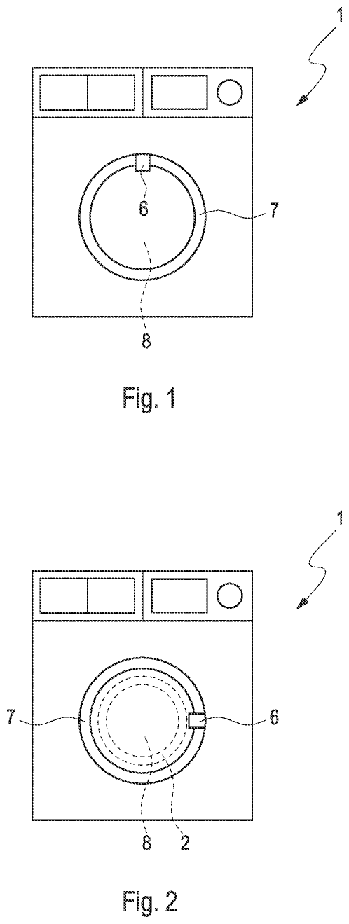

FIG. 1 shows a washing machine 1 with a viewing window 7 and an interior washing compartment 8 in which the laundry is cleaned. Furthermore, a locking device 6 is present, which ensures that the laundry is only irradiated with an ultraviolet radiation source, if the washing compartment 8 is closed.

FIG. 2 shows a washing machine 1 with a viewing window 7. Inside the washing compartment 8 of the washing machine 1 an ultraviolet radiation source 2 is arranged, which can disinfect the laundry with ultraviolet radiation or UV-C radiation during a washing process. By means of the ultraviolet radiation pathogenic germs, viruses or bacteria can be killed. The washing machine 1 can be formed in such a way, that the ultraviolet radiation source 2 can only be switched on, if the locking device 6, which is formed as a device for determining whether the washing compartment 8 is closed. In addition, the ultraviolet radiation source 2 can be insulated against the outside world by a material, that reliably prevents ultraviolet radiation from escaping to the outside. Glass or plastic, that can absorb the ultraviolet radiation, can in particular be used for this purpose. The ultraviolet radiation source 2 can be circular in shape in order to better be able to illuminate and disinfect the laundry with ultraviolet radiation.

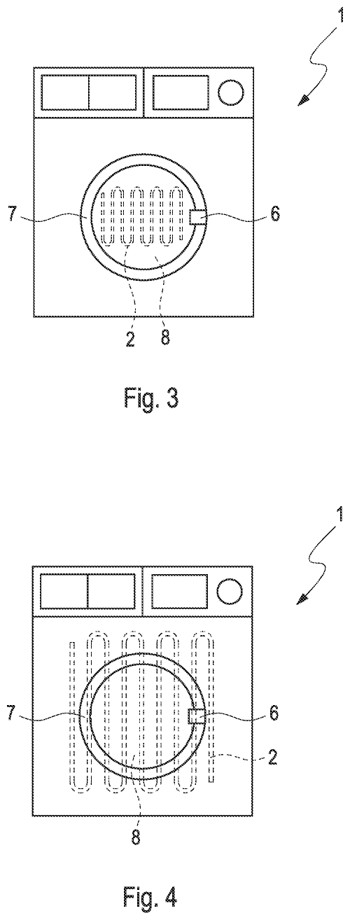

FIG. 3 shows a washing machine according to the invention with a meander shaped ultraviolet radiation source 2. As a result, an evenly strong ultraviolet radiation is shone onto the items to be washed. The ultraviolet radiation source 2 is arranged inside the washing compartment 8 of the washing machine in order to enable direct irradiation of the ultraviolet radiation source 2 onto the items to be washed. In an alternative embodiment, a material transparent to the ultraviolet radiation is arranged between the items to be washed and the ultraviolet radiation source 2.

FIG. 4 shows a washing machine according to the invention with a meander shaped ultraviolet radiation source 2. The ultraviolet radiation source 2 is formed in a planar manner in such a way that ultraviolet radiation can be radiated over the entire front surface of the washing compartment 8 into the interior of the washing compartment 8.

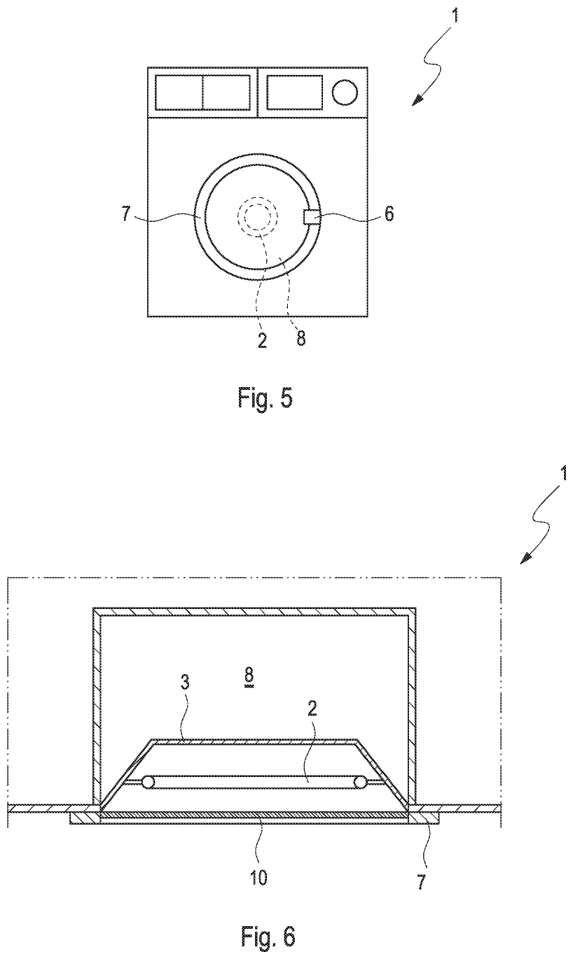

FIG. 5 shows an alternative washing machine 1 according to the invention with a circular ultraviolet radiation source 2.

FIG. 6 shows, in a cross-sectional view from above, the washing compartment 8, in which the laundry can be placed, and the ultraviolet radiation source 2, which can emit ultraviolet radiation into the washing compartment 8 in such a way that germs, bacteria and viruses are killed. A material 10, which absorbs the ultraviolet radiation, is arranged for protection for the outside world. Said material 10 can be glass, plastic or perforated sheet metal. In addition, another material 3 can be arranged inside the washing compartment 8 in such a way, that the laundry cannot reach the ultraviolet radiation source 2. This ensures, that individual parts of the laundry cannot get tangled on the ultraviolet radiation source and thereby prevent irradiation of the entire laundry. The material 3 is transparent to ultraviolet radiation. The material 3 can consist of synthetic quartz glass, glass or plastic, for example. In an alternative embodiment, the material 10 can be a section of the viewing window 7.

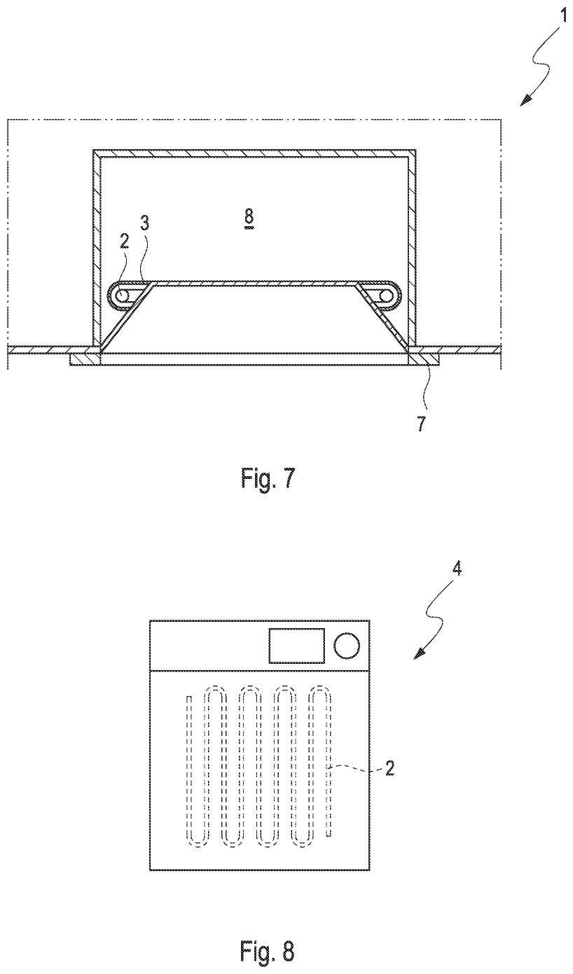

FIG. 7 shows a cross-section through an alternative embodiment of a washing machine according to the invention, wherein inside the washing compartment 8 an ultraviolet radiation source 2 is arranged, which is separated from the items to be washed by a material 3, which is designed as an encapsulation for the ultraviolet radiation source 2. By means of the viewing window 7, the ultraviolet radiation from the ultraviolet radiation source 2 is shielded against the outside world, since the viewing window consists of a material that absorbs UV-C or ultraviolet radiation.

FIG. 8 shows a dishwasher 4 with a meander-shaped radiation source 2, which is arranged inside the washing compartment of the dishwasher 4. In an alternative embodiment of the dishwasher, the ultraviolet radiation source 2 is not separated from the items to be washed by a material, since, in contrast to a washing machine, the items to be washed in a dishwasher are not thrown around in a washing drum. Therefore, there is no need to fear that items to be cleaned will cling to the ultraviolet radiation source and cover the ultraviolet radiation source 2, thereby preventing uniform irradiation of the items to be washed.

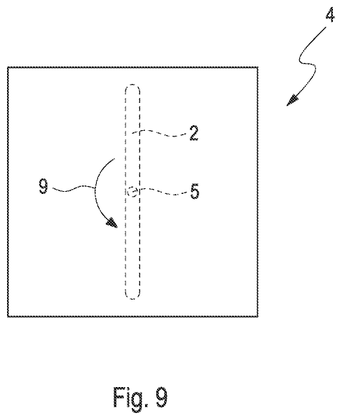

FIG. 9 shows an alternative embodiment of a dishwasher 4 according to the invention with a movable ultraviolet radiation source 2, which is turnable or rotatable about an axis 5, for example. As a result, better illumination of the items to be washed in a dishwasher 4 can be achieved. This is particularly advantageous because the items to be washed in a dishwasher 4 are not moved within the washing compartment. In an even more advantageous embodiment, the movable ultraviolet radiation source 2 interacts with a reflector arranged inside the washing compartment or at the edge of the washing compartment. As a result, the ultraviolet radiation can be shone from any point onto the items to be washed.

In a particular embodiment, the interior of the washing machine or dishwasher is lined with material that reflects UV light, so that pathogenic germs, bacteria and viruses can be killed by the reflected UV light.

In a further particular embodiment, the washing machine or dishwasher has two, three or four UV light sources, which are arranged in such a way that different areas of the washing machine or dishwasher are illuminated.

In yet another particular embodiment, the washing machine or the dishwasher has a first UV light source illuminating the washing compartment from above, and/or a second UV light source illuminating the washing compartment from below, and/or a third UV Light source illuminating the washing compartment from the left, and/or a fourth UV light source illuminating the washing compartment from the right, and/or a fifth UV light source illuminating the washing compartment from the front, and/or a sixth UV-Light source illuminating the washing compartment from behind.

UV light source to kill the corona and other viruses, bacteria. Illuminating from the outside in.

The invention is particularly suitable for killing corona viruses. For this purpose, a UV light source is used, which can illuminate into the washing compartment of a washing machine or dishwasher.

UV-C transparent glass. Advantageously, the washing compartment is separated from the ultraviolet radiation source by a UV-C transparent glass.

Washing machine door closed and light source on (open light source off).

Advantageously, the ultraviolet radiation source can only be switched on, if the washing compartment is locked.

In an alternative embodiment, a UV light source/ultraviolet radiation source is arranged on the outside of the washing machine door, which can emit its UV radiation inwards. As a result, bacteria and germs can be killed during the washing process by the UV light shining into the drum/washing compartment.

The invention relates to combating viruses and bacteria in washing machines by means of UV light.

It is known from the prior art, that there have been various attempts to combat viruses and bacteria in washing machines. If UV light is used directly, it is harmful to your eyes.

If an UV light source were to be placed on the outside of the door and the UV light source shines into the inside of the washing machine drum, this danger is eliminated.

The lamp is only on when the door is closed. In addition, changing this lamp is easier.
1. UV light source attached to the outside of the washing machine door.
2. UV light source shining inside the drum.
3. Door closed and UV light source on.
4. Door open and UV light source off.

It shall be noted that the term “comprising” does not preclude other elements or steps, just as the terms “a” and “an” do not preclude multiple elements and steps.

The reference numbers used are for convenience only and should not be taken as limiting in any way, the scope of the invention being indicated by the claims.

1.-10. (canceled)

11. A washing machine or dishwasher comprising:
a washing compartment configured to clean laundry or dishes; and
an ultraviolet radiation source configured to irradiate the laundry or the dishes,
wherein the ultraviolet radiation source is arranged within the washing compartment.

12. The washing machine or dishwasher according to claim 11, wherein the ultraviolet radiation source is separated from the washing compartment by a first material, which is transparent to ultraviolet radiation, and/or wherein the first material is glass or plastic.

13. The washing machine or dishwasher according to claim 12, wherein the first material comprises synthetic quartz glass.

14. The washing machine or dishwasher according to claim 11, wherein the ultraviolet radiation source is movable in order to scatter ultraviolet radiation of the ultraviolet radiation source arbitrarily within the washing compartment during washing or cleaning.

15. The washing machine or dishwasher according to claim 14, wherein the ultraviolet radiation source is turnable or rotatable.

16. The washing machine or dishwasher according to claim 11, wherein the ultraviolet radiation source comprises:
a first ultraviolet radiation source configured to illuminate the washing compartment from above, and/or
a second ultraviolet radiation source configured to illuminate the washing compartment from below, and/or
a third ultraviolet radiation source configured to illuminate the washing compartment from left, and/or
a fourth ultraviolet radiation source configured to illuminate the washing compartment from right, and/or
a fifth ultraviolet radiation source configured to illuminate the washing compartment from front, and/or
a sixth ultraviolet radiation source configured to illuminate the washing compartment from behind.

17. The washing machine or dishwasher according to claim 11, wherein the ultraviolet radiation source comprises:
a first ultraviolet radiation source configured to illuminate the washing compartment from above,
a second ultraviolet radiation source configured to illuminate the washing compartment from below,
a third ultraviolet radiation source configured to illuminate the washing compartment from left,
a fourth ultraviolet radiation source configured to illuminate the washing compartment from right,
a fifth ultraviolet radiation source configured to illuminate the washing compartment from front, and
a sixth ultraviolet radiation source configured to illuminate the washing compartment from behind.

18. The washing machine or dishwasher according to claim 11, further comprising a reflector arranged in the washing compartment so that ultraviolet radiation from the ultraviolet radiation source is reflectable back into the washing compartment.

19. The washing machine or dishwasher according to claim 18, wherein the reflector is movable in order to scatter the ultraviolet radiation arbitrarily during washing or cleaning.

20. The washing machine or dishwasher according to claim 19, wherein the reflector is turnable or rotatable.

21. The washing machine or dishwasher according claim 11, wherein the washing compartment is lined with a second material configured to reflect ultraviolet radiation.

22. The washing machine or dishwasher according to claim 11, further comprising a device configured to determine whether the washing compartment is closed, wherein the ultraviolet radiation source is only switched on when the device signals a closed washing compartment.

23. The washing machine or dishwasher according to claim 11, wherein the ultraviolet radiation source is circular, or semicircular or meander shaped.

24. The washing machine or dishwasher according to claim 11, wherein a third material is located between an outer area of the washing machine or dishwasher and the ultraviolet radiation source, and wherein the third material is configured to absorb ultraviolet radiation.

25. The washing machine or dishwasher according to claim 24, wherein the third material is glass, perforated sheet metal or plastic.

26. The washing machine or dishwasher according to claim 11, wherein the ultraviolet radiation source is a UV-C radiation source.
